(19) United States
(12) Patent Application Publication
(10) Pub. No.: US 2025/0031977 A1
(43) Pub. Date: Jan. 30, 2025

(54) SYSTEMS AND METHODS FOR MEASURING A RESPONSE OF A SUBJECT TO AN EVENT

(71) Applicant: Oxford University Innovation Limited, Oxford (GB)

(72) Inventors: Rebeccah SLATER, Oxford (Oxfordshire) (GB); Aomesh BHATT, Oxford (Oxfordshire) (GB); Kirubin PILLAY, Oxford (Oxfordshire) (GB); Caroline HARTLEY, Oxford (Oxfordshire) (GB); Simon MARCHANT, Oxford (Oxfordshire) (GB)

(21) Appl. No.: 18/709,043

(22) PCT Filed: Nov. 9, 2022
(86) PCT No.: PCT/GB2022/052836

§ 371 Date: May 10, 2024

(30) Foreign Application Priority Data
Nov. 11, 2021 (GB) 2116218.5

(51) Int. Cl. A61B 5/0205; A61B 5/00; A61B 5/024; A61B 5/372; A61B 5/383; G16H 50/30

(52) U.S. Cl. CPC A61B 5/0205; A61B 5/024; A61B 5/372; A61B 5/383; A61B 5/7246; A61B 5/7264; A61B 5/7271; G16H 50/30

(57) ABSTRACT

The invention relates to a method (30) comprising: acquiring (32), using an EEG monitoring system (12), EEG data from a subject (16), said data being recorded over a first time period, said first time period including an event; acquiring (34), using a heart rate monitoring system (14), heart rate data from the subject (16), said heart rate data being recorded over a second time period, said second time period also including the event; scaling (36) an EEG template to fit event EEG data at a specified latency following the event to derive an EEG scaling factor; determining (38) a heart rate change due to the event using the heart rate data; and combining (40) the EEG scaling factor and the heart rate change to generate a score indicative of a response of the subject to the event.

### TECHNICAL FIELD

The present invention relates to systems and methods for measuring a response of a subject to an event, and in particular to systems and methods for quantifying pain experienced by a subject in response to an event, such as a tactile or noxious stimulus. The present invention relates particularly, but not exclusively to methods and systems for quantifying pain experienced by neonates and infants in response to a noxious stimulus.

### BACKGROUND

Hospitalised neonates undergo painful procedures each day, including heel lances, cannulations and peripheral line insertions, however it is difficult to measure their pain or to test whether analgesics are effective. Neonatal pain assessment primarily relies on various behavioural indicators, such as changes in facial expression and variation in vital signs, like heart rate. These tools are inherently subjective and/or non-specific.

There is a need for tools that more accurately evaluate pain in neonates. While more than 40 different composite pain scales have been developed to assess neonatal pain there is a clear acknowledgement that better pain measurement tools are required in clinical practice and clinical trials. Currently multidimensional scales, which are the accepted standard for neonatal pain assessment, are limited because they have been developed empirically and lack key aspects of objectivity, specificity, and validity. Further, behavioural and physiological observations that are widely used to assess pain primarily rely on motor or autonomic processes that are not directly linked to pain experience.

Well-characterised patterns of noxious-evoked brain activity have been recorded in response to acute nociceptive procedures (such as heel lancing, cannulation, and intramuscular injections) in neonates aged 34-42 weeks' gestation. However, although it has been demonstrated that it is possible to identify EEG activity resulting from a noxious stimulus, as yet a system and method which is able to quantify the pain intensity experienced by a wider age range in a manner which is comparable between patients has yet to be developed.

### SUMMARY OF THE INVENTION

According to a first aspect of the invention, we provide a method comprising:
acquiring, using an EEG monitoring system, EEG data from a subject, said data being recorded over a first time period, said first time period including an event;
acquiring, using a heart rate monitoring system, heart rate data from the subject, said heart rate data being recorded over a second time period, said second time period also including the event;
scaling an EEG template to fit event EEG data at a specified latency following the event to derive an EEG scaling factor;
determining a heart rate change due to the event using the heart rate data; and
combining the EEG scaling factor and the heart rate change to generate a score indicative of a response of the subject to the event.

“Event EEG data” as used herein means EEG data which has been acquired from the subject and which can be attributed as being responsive to the event. EEG data may be attributed as responsive to an event if it occurs at a specified latency following the event, such that the event can be said to have caused the electrical signals that are recorded as EEG data. As discussed in further detail below, the specified latency may be determined from experimental data.

The score provides a measure of the physical (EEG and heart rate) response of the subject to an event. Thus, the score may be useful to a clinician who is assessing the response of the subject to the event. For example, where the response is a pain response, a clinician may use the score to assess if pain is felt by a subject in response to the event, and if so, how much pain. The score thus provides a measure of pain that can be helpful to a clinician in diagnosing the subject with a medical condition that may be the cause of said pain.

The method may further comprise selecting the EEG template from a set of age-dependent templates to obtain an age-appropriate EEG template, said selection being based on an age of the subject. EEG data acquired from a subject can vary in dependence on the age of the subject. Thus, the accuracy of the score may be improved if the template that is used to scale the event EEG data is derived from EEG data recorded from subjects having the same or a similar age to the subject being assessed. That is, the age of the subject being assessed may preferably fall within an age range, wherein the template is derived from data acquired from subjects within that age range.

The selection of the template may be made using a weighted probability function. Use of a weighted probability function in template selection may improve the accuracy of the score in cases where the age of the subject being assessed falls within a boundary age range.

The method may further comprise: deriving a goodness-of-fit between the EEG template and the event EEG data; and weighting the EEG scaling factor using the goodness-of-fit to produce a weighted EEG scaling factor. Weighting the EEG scaling factor using goodness-of-fit improves accuracy of the score by reducing the likelihood of spurious EEG signals resulting in a high score.

The method may further comprise: acquiring, using the EEG monitoring system, baseline EEG data from the subject, said baseline EEG data being recorded over a plurality of baseline EEG time periods prior to the event; and modulating the EEG scaling factor using the baseline EEG data to obtain a modulated EEG scaling factor. Modulating the EEG scaling factor may comprise: scaling the EEG template to fit the baseline EEG data to produce scaled baseline EEG data for each of said baseline EEG time periods; and modulating the EEG scaling factor using the scaled baseline EEG data to obtain the modulated EEG scaling factor. Modulation of the EEG scaling factor with baseline data permits scores from different individuals to be compared.

The method may further comprise:
deriving, for each of the plurality of baseline EEG time periods, a goodness-of-fit between the EEG template and the scaled baseline EEG data;
weighting, for each of the plurality of baseline EEG time periods, the scaled baseline EEG data using the derived goodness-of-fit to produce weighted scaled baseline EEG data;
calculating the mean and the standard deviation of the weighted scaled baseline EEG data; and
standardising the EEG scaling factor using:

$${API}_{EEG , i} = \frac{{ϵ}_{i} {r}_{i} - μ}{σ} \qquad ( 1 )$$

where ϵᵢ is the EEG scaling factor for the event, rᵢ is the goodness-of-fit between the EEG template and the event EEG data, μ is the mean of the weighted scaled baseline EEG data, and σ is the standard deviation of the weighted scaled baseline EEG data. Standardising the EEG scaling factor using baseline data results in a score that is subject-invariant (as regards EEG data), allowing direct comparisons to be made of different subjects' responses to similar events.

The method may further comprise: acquiring, using the heart rate monitoring system, baseline heart rate data from the subject, said baseline heart rate data being recorded over a plurality of baseline heart rate time periods prior to the event; and modulating the heart rate change due to the event using the baseline heart rate data. The method may further comprise: determining a pre-event heart rate change for each of the plurality of baseline heart rate time periods; and standardising the heart rate change due to the event by subtracting the mean of the pre-event heart rate changes from the heart rate change due to the event and dividing the result by the standard deviation of the pre-event heart rate changes. Standardising the heart rate change using baseline data results in a score that is subject-invariant (as regards heart rate), allowing direct comparisons to be made of different subjects' responses to similar events.

The step of combining the standardised EEG scaling factor and the standardised heart rate change to generate a score may comprises:
defining a first threshold function based on the standardised heart rate change and a second threshold function based on the standardised heart rate change,
leaving the score unchanged if the standardised EEG scaling factor falls between the first and second threshold functions,
decreasing the score if the standardised EEG scaling factor is greater than the first threshold function, and
increasing the score if the standardised EEG scaling factor is less than the second threshold function.

Threshold functions of the type discussed above can reduce the effect of outlying results on the score—e.g., very high or low heart rate changes have less influence on the score than they otherwise would do.

The method may further comprise discretising the score.

The event may be a tactile stimulus and/or a noxious stimulus.

According to a second aspect of the invention, we provide a system for quantifying pain experienced by a subject in response to an event, the system comprising:
an EEG monitoring system operable to acquire EEG data from the subject;
a heart rate monitoring system operable to acquire heart rate data from the subject; and
a processor operable to:
   acquire, using the EEG monitoring system, EEG data from a subject, said data being recorded over a first time period, said first time period including an event;
   acquire, using the heart rate monitoring system, heart rate data from the subject, said heart rate data being recorded over a second time period, said second time period also including the event;
   scale an EEG template to fit event EEG data at a specified latency following the event to derive an EEG scaling factor;
   determine a heart rate change due to the event using the heart rate data; and
   combine the EEG scaling factor and the heart rate change to generate a score indicative of a response of the subject to the event.

The processor may be configured to carry out the method of the first aspect of the invention using data acquired by the EEG monitoring system and heart rate monitoring system.

According to a third aspect of the invention, we provide a computer programme product operable, when run on a processor in signal communication with an EEG monitoring system and a heart rate monitoring system, to cause the processor to:
acquire, using the EEG monitoring system, EEG data from a subject, said data being recorded over a first time period, said first time period including an event;
acquire, using the heart rate monitoring system, heart rate data from the subject, said heart rate data being recorded over a second time period, said second time period also including the event;
scale an EEG template to fit event EEG data at a specified latency following the event to derive an EEG scaling factor;
determine a heart rate change due to the event using the heart rate data; and
combine the EEG scaling factor and the heart rate change to generate a score indicative of a response of the subject to the event.

The computer programme product may be operable to run on the processor of the second aspect of the invention, and/or to carry out the method of the first aspect of the invention.

It will be appreciated that features from the second and third aspects of the invention may be combined with features from the first aspect of the invention, either separately or in combination, if required. Features from any aspect of the invention may be combined with features set out in the description below, either in combination with other features or in isolation.

### BRIEF DESCRIPTION OF THE DRAWINGS

The invention will now be described, by way of example only, with reference to the accompanying drawings, in which:

FIG. 1 shows a schematic overview of a system for measuring a response of a subject to an event;

FIG. 2 illustrates a method for measuring a response of a subject to an event, where items depicted in dashed outline are optional;

FIGS. 2A and 2B illustrate example templates;

FIG. 3 illustrates one approach for modulating the score;

FIG. 4 shows sensitivity and specificity for discriminating ‘pain’ from ‘no pain’ at different thresholds of the score;

FIG. 5 illustrates one option for template selection;

FIG. 6 illustrates an alternative option for template selection;

FIG. 7 sets out an overview of the mathematical steps involved in an example implementation of the method of FIG. 2;

FIG. 8 illustrates the development and testing of a preterm template;

FIG. 9 shows a receiver operating characteristic (ROC) curve for the discrimination between noxious and non-noxious stimuli;

FIG. 10 shows average EEG template coefficients (left) and median scores (right) in response to common acute clinical procedures and a non-noxious control;

FIG. 11 shows scores following experimental noxious stimulation applied to a group of neonates (n=10 participants) without anaesthetic (Control) and with local anaesthetic applied prior to the stimulation (Local anaesthetic);

FIG. 12 shows individual neonates scores in response to experimental noxious stimulation applied before and after the application of local anaesthetic;

FIG. 13 shows that scores following immunisation are significantly lower in a group of neonates who received paracetamol compared with a group of infants who did not receive paracetamol prior to the immunisations; and

FIG. 14 shows that neonates with infection in the first days of life (infected group) present significantly higher scores compared with healthy neonates (uninfected group).

### DETAILED DESCRIPTION

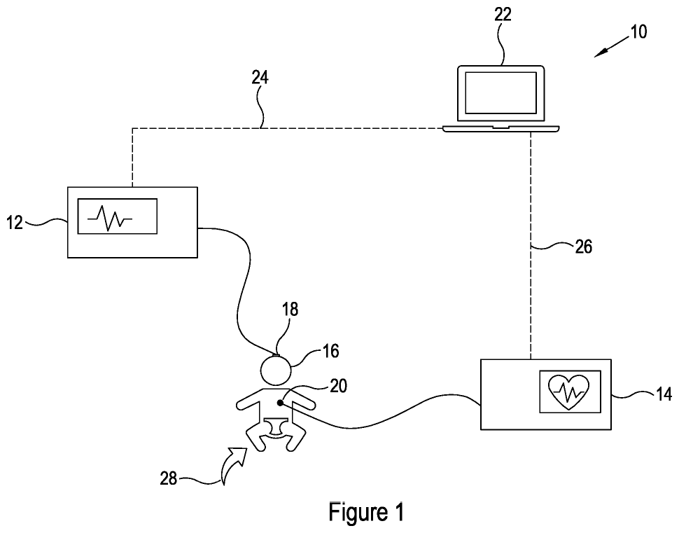

Referring first to FIG. 1, a system 10 is shown. The system includes an electroencephalography (EEG) monitoring system 12 and a heart rate monitoring system 14. The EEG monitoring system 12 is operable to acquire EEG data from a subject 16, in this case an infant. Similarly, the heart rate monitoring system 14 is operable to acquire heart rate data from the subject 16.

The EEG monitoring system may be any system capable of obtaining EEG data signals from a brain of a subject. Typically such systems include at least one recording electrode (in addition to a reference and ground electrode) that, when placed against the scalp of a wearer, is able to detect electrical activity due to brain activity of the wearer in a known manner (electrical activity recorded in response to stimuli is known as evoked potentials). In the example shown in FIG. 1, the EEG monitoring system 12 includes at least a Cz electrode 18, which is shown attached to the scalp of the subject.

Similarly, the heart rate monitoring system 14 may be any system capable of obtaining heart rate data signals from a subject. In the example shown in FIG. 1, the heart rate monitoring system 14 is an electrocardiography (ECG) system. The system includes at least one recording electrode 20 that, when placed on the skin of the subject, is operable to detect electrical signals produced by the heart. Heart rate may be calculated from such signals in a known manner.

The system 10 further includes a processor 22. The processor is operable to receive EEG data signals 24 from the EEG monitoring system 12 and to receive heart rate data signals 26 from the heart rate monitoring system 14. The processor may be local to (e.g. in the same room or building as) the EEG monitoring system and heart rate monitoring system. However, this is not essential, and the processor may be remote from the EEG monitoring system and heart rate monitoring system (for example a cloud-based server). The received signals are used by the processor 12 to generate a score, referred to herein as the Acute Pain Index (API).

The API score is indicative of a response of the subject 16 to an event, indicated schematically in FIG. 1 by arrow 28. The event may be a painful (noxious) event, such as a heel lance or other medical procedure. Alternatively, the event may be a tactile event, that may be mildly noxious or non-noxious. In either case the API score quantifies the pain response of the individual subject to the event. This gives a measure of the pain experienced by the subject in response to the event.

As will be explained in more detail below, the API score is validated for use in (i) premature neonates (from 29-36 weeks' gestation), (ii) term-aged neonates (from 37-42 weeks' gestation) and (iii) infants up to 6 months of age. The API combines EEG and heart rate signals to infer the level of pain that a baby is experiencing while they are at rest during a tactile stimulus or in response to a mild noxious stimulus, or in response to an acute painful procedure (e.g. an immunisation, cannulation or heel lance).

The API is calculated using a machine-learning approach to analyse EEG and ECG signals that are evoked in response to a tactile (non-noxious or mildly noxious) stimulus applied to the surface of the skin while the infant is at rest, or by an acute painful clinically-required procedure. When the tactile (mild noxious) stimulus is applied, the API can also be used to infer the infant's ongoing pain sensitivity.

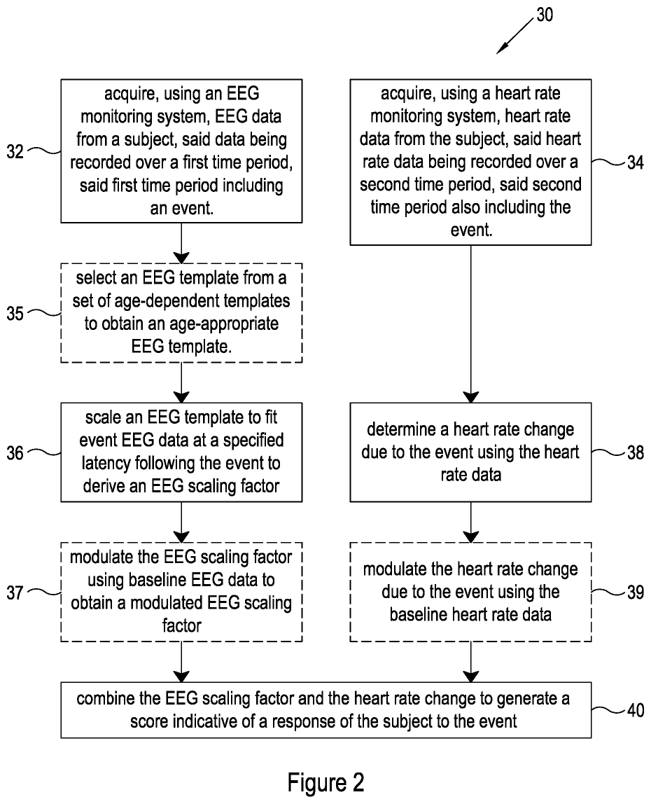

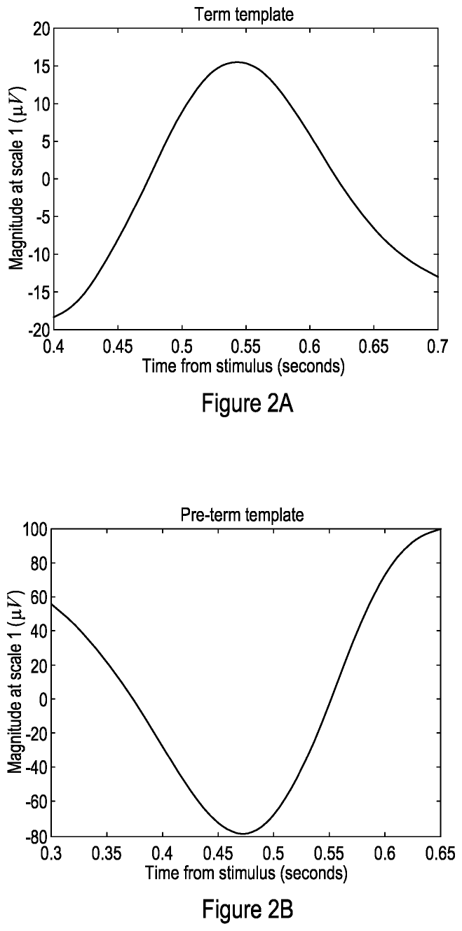

Referring to FIG. 2, a method for quantifying the pain experienced by a subject in response to an event is illustrated generally at 30. The method may be carried out by a system of the type shown in FIG. 1.

Item 32 includes acquiring, using an EEG monitoring system such as the EEG monitoring system 12, EEG data from a subject 16. The EEG data is recorded over a first time period, which first time period includes an event of the type discussed above, such as a stimulus (e.g., noxious or mild noxious tactile stimulus).

Similarly, item 34 includes acquiring, using a heart rate monitoring system such as the ECG system 14, heart rate data from the subject. The heart rate data is recorded over a second time period, which second time period also includes the event.

The first and second time periods necessarily overlap (since both include the same event 28), and may be the same time period, although that is not necessary. The EEG and heart rate data are acquired as electrical signals and may be transmitted to the processor via wired or wireless connection.

Following EEG data acquisition, item 36 includes scaling an EEG template to fit event EEG data at a specified latency following the event to derive an EEG scaling factor. The template may be selected from a set comprising a plurality of possible templates, as indicated by optional item 35.

Similarly, following heart rate data acquisition, item 38 includes determining a heart rate change due to the event using the heart rate data.

The EEG scaling factor and the heart rate change are then combined, in item 40, to generate a score (or API) indicative of a response of the subject to the event. Prior to any such combination the EEG scaling factor and the heart rate change may be normalised so that the score can be standardised across individuals, as indicated by optional items 37 and 39. The resulting score may be scaled between 0 and 10 to calculate a pain score which classifies pain according to the following categories: no pain (0), mild pain (1-3), moderate pain (4-8) and severe pain (9-10).

A detailed discussion of an exemplary process for calculating a score according to the method outlined above will now follow, discussing first the EEG data processing method, second the heart rate data processing method, and finally the combination of the processed EEG data and processed heart rate data to generate an API score.

The input data for an infant is received at the processor 22 as a multidimensional array containing both an EEG recording (e.g. taken from a single electrode, such as a Cz electrode) and an ECG recording made during application of a stimulus, such as a noxious stimulus. The recording may include multiple events, e.g. multiple applications of a stimulus, which may be the same stimulus (e.g. in the case of a stimulus such as a mild noxious stimulus) or differing stimuli (e.g. such as a control stimulus followed by a clinically necessary noxious stimulus). Both the EEG and ECG contain multiple timestamps for all applications of a stimulus and each application is known as a recording event. It is therefore possible to determine an event time (that is, a time at which a stimulus event occurred) from the received data.

In prior work we have shown that an EEG recording from a subject typically shows a waveform which can be characterised as a pain response between 400 ms and 700 ms following a noxious event in term infants. The EEG recording is therefore made over a first time period which is long enough to include such a pain response. Similarly, the ECG recording is made over a second time period which is long enough to include any heart rate response to the event. As will be discussed in more detail below, we have found that it is also useful to record background (i.e. pre-event) data. For this reason the first and second time periods may include pre-event data as well as post-event data. For EEG and ECG the recording is epoched around each event that includes a baseline pre-event period and a post-event period (for example, one minute before and after the event).

Prior to any API calculation the signal may be pre-processed to remove noise. In one example method, the EEG is bandpass filtered in the range 0.5-30 Hz, and the ECG in the range 12-40 Hz. Both signals are notch-filtered at 50 Hz and 60 Hz to remove mains noise (UK and USA, respectively) and then resampled to 2000 Hz to standardise the API calculations. In addition, the EEG is baseline-corrected by subtracting the mean of the pre-event baseline immediately before the event. This is standard practise for pre-filtering signals that are analysed from clinical monitors.

### EEG Signal Processing

### 1. Template Projection

This initial stage of calculating API involves fitting or projecting a pre-determined template onto the EEG data at a specified latency after the stimulus is applied. The “template” may be in the form of an EEG signal that is characteristic of a pain response. As will be discussed in more detail below, the template may be derived from data gathered from individuals that are known to be experiencing pain. The template may have both a shape and a length that is characteristic of a pain response.

FIG. 2A shows an example template derived from term infants (aged 37-42 weeks). FIG. 2B shows an example template derived from pre-term infants (aged 29-34 weeks). Each template comprises a start time and an end time, that together define a time window. For the term template the start time is approximately 400 ms from the stimulus, and the end time is approximately 700 ms from the stimulus, defining a time window of length approximately 300 ms. For the term template the start time is approximately 300 ms from the stimulus, and the end time is approximately 650 ms from the stimulus, defining a time window of length approximately 350 ms. Within the time window the templates each have a shape that has been found to be characteristic of a pain response. It will be appreciated that other shapes and time windows may be appropriate for subjects of a different age—such templates may be derived from experimental data as discussed in more detail below.

Fitting the template may thus comprise digitally overlaying and scaling the template on the recorded EEG signal in order to determine how closely the recorded data matches the template data. The more similar the recorded data is to the template, the more likely it is that the subject is experiencing pain.

The latency may be approximately 400-700 ms in the case of term infants, and 300-650 ms in the case of pre-term infants, such that the template is fitted to the EEG signal at a time period during which the subject is likely to be experiencing a response to the event. The latency varies depending on the age of the infant and the template used.

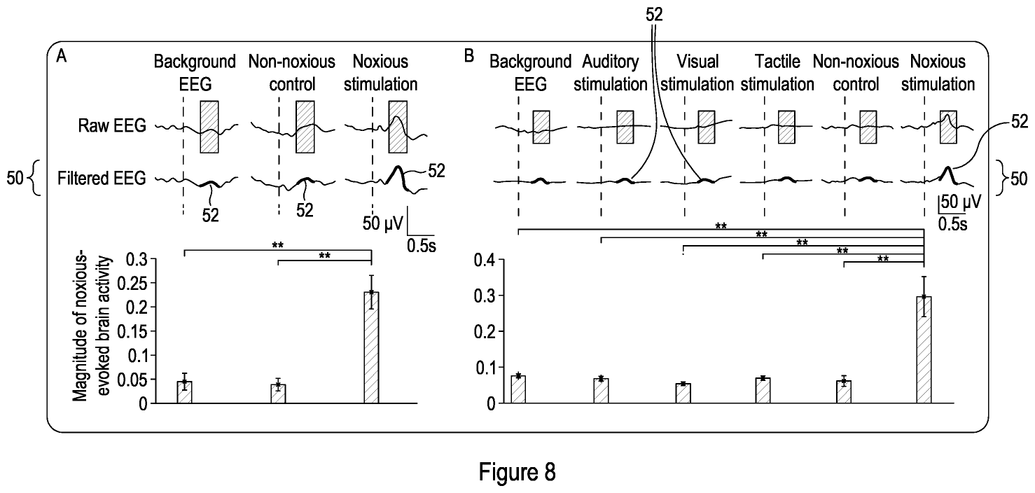

Examples showing how a template is fitted to recorded EEG data are illustrated in FIG. 8, and are discussed in more detail below.

Prior to fitting the template to the pre-processed EEG data a Woody Filtering of the EEG signal may be performed to better temporally align the EEG with the template. Woody filtering maximises the cross-correlation between the signal up to a maximum shift, or ‘jitter’, of 100 ms.

As one option for scaling the template to fit the EEG data, Singular Value Decomposition (SVD) of the template may be performed and the resulting decomposed matrices used to re-scale the template to best fit the woody-filtered EEG.

Alternatively, instead of performing SVD to scale the template, it is possible to use a direct application of ‘least-squares’ to create an inversion vector, x, that is then stored and reused by the algorithm.

Defining a general template as v:

$$x = {( {v}^{T} v )}^{- 1} {v}^{T} \qquad ( 2 )$$

A scaling factor for each i^{th} event, ϵᵢ, is then determined by multiplying the inversion vector with the corresponding woody-filtered EEG data, bᵢ:

$${ϵ}_{i} = {b}_{i} x \qquad ( 3 )$$

This approach is more computationally efficient than an SVD method.

### 2. Goodness-of-Fit

The template scaling factor c, represents a measure of the magnitude of the subject's EEG response to the event. However, we have found that on occasion spurious signals may be incorrectly characterised by the template as pain-evoked activity. We have therefore found it helpful to calculate a goodness-of-fit between the template and the EEG data to derive a weighted EEG scaling factor.

For example, goodness-of-fit may be determined using Pearson's correlation coefficient, rᵢ, between the template and the woody-filtered EEG. The correlation metric for goodness-of-fit can be used to adjust the API output if the template fit is poor.

Using the outputs of the template projection stage, a goodness-of-fit-weighted EEG scaling factor is derived, which penalizes the value if the template fit is poor. An example of such a weighted scaling factor, using Pearson's coefficient, is:

$${\tilde{ϵ}}_{ι} = {ϵ}_{i} {r}_{i} \qquad ( 4 )$$

### 3. Correction for Intra-Patient Variability

The above discussed EEG scaling factor is patient-specific. It can thus be beneficial to produce a standardised EEG scaling factor which is comparable between patients. To produce such a standardised EEG scaling factor (also referred to as API_{EEG}) background EEG activity is used to modify the amplitude of the template so that EEG scaling factors are standardised and comparable between participants, so correcting for intra-patient variability in background EEG activity.

Such a correction may be performed by first acquiring, using the EEG monitoring system, baseline EEG data from the subject, said baseline EEG data being recorded over a plurality of baseline EEG time periods prior to the event. The EEG scaling factor may then be modulated using the baseline EEG data to obtain a modulated EEG scaling factor.

In particular, the EEG template may be scaled to fit the baseline EEG data in the same manner as that described above to produce scaled baseline EEG data for each of the baseline EEG time periods. Goodness-of-fit may also be determined for each of the baseline scaling factors, and used to weight those scaling factors, as discussed above. The above template projection and goodness-of fit-weighting steps may be repeated across multiple windows of background EEG.

The mean (μ) and standard deviation (σ) of these baseline values are calculated as standardizing metrics.

### 4. Standardised EEG Scaling Factor

Finally, a standardised EEG scaling factor API_{EEG,i}, for an event i, may be determined by the following equation:

$${API}_{EEG , i} = \frac{{\overbrace{{ϵ}_{i} {r}_{i}}}^{{\tilde{ϵ}}_{ι}} - μ}{σ} \qquad ( 5 )$$

where ϵᵢ is the EEG scaling factor for the event, rᵢ is the goodness-of-fit between the EEG template and the event EEG data, μ is the mean of the weighted scaled baseline EEG data, and σ is the standard deviation of the weighted scaled baseline EEG data.

This measure is now invariant to infant-specific differences to the background EEG and poor template fits.

### Heart Rate Signal Processing

### 1. Heart Rate Change

In tandem with the EEG calculations, the heart rate is derived. In this example heart rate is derived from the pre-filtered ECG signal. The approach for deriving heart rate from an ECG signal is standard practise and involves the following steps:
1. Perform R-peak detection of the ECG using an R-peak detection algorithm. In the present example we used the (open-source) Engzee peak-detection approach.
2. Generate the RR-interval signal (difference between successive R-R peaks).
3. Derive the heart rate signal by averaging the RR-interval signal within 3 s windows (Is shifts) and taking the inverse.

From the heart rate signal, a metric to describe the change in HR due to the i^{th} event, ΔHRᵢ, is determined by subtracting the maximum heart rate value in the 10 s post-stimulus from the mean heart rate value in the 5 s pre-stimulus. This is denoted as:

$$Δ {HR}_{i} {\vert}_{t = 0} = max [ {HR ( t )}_{i} {\vert}_{t = 0 : 10 s} ] - mean [ {HR ( t )}_{i} {\vert}_{t = - 5 : 0 s} ] \qquad ( 6 )$$

where the event time is assumed to be at t=0 s.

### 2. Correction for Intra-Patient Variability

As with the EEG scaling factor discussed above, the heart rate change due to the event may be modulated with background heart rate information in order to correct for intra-patient variability.

In one example such a correction may be achieved by repeating the above heart rate change determination steps (1)-(3) in is shifts in the minute of background ECG prior to the first event. ΔHRᵢ is normalised to this distribution to standardize the ΔHRᵢ measure and reduce intra-patient variability:

$${API}_{HR , i} = \frac{Δ {HR}_{i} {\vert}_{t = 0} - Δ {HR}_{j = 1} {\vert}_{t = T - 55 : T - 10}}{SD ( Δ {HR}_{j = 1} {\vert}_{t = T - 55 : T - 10}} \qquad ( 7 )$$

where T=time of first event.

For example, in the background period each background value is calculated around a background time stamp (5 s pre- to 10 s post-time stamp), and this background time stamp, t, is shifted in 1 s intervals across the 1 minute prior to the stimulus event. If, as in this example, the calculation considers the 5 s pre the background time stamp, then the earliest time it can start in the one minute prior to the stimulus is at T−55 s and the latest it can reach (without exceeding the stimulus) is t−10 s.

The above equation can alternatively be written:

$${API}_{HR , i} = \frac{Δ {HR}_{i} - {μ}_{HR}}{{σ}_{HR}} \qquad ( 8 )$$

where ΔHRᵢ is the heart rate change for the event, μ_{HR} is the mean of the baseline heart rate change data, and σ_{HR} is the standard deviation of the baseline heart rate change data.

### API Score Calculation

To calculate the final API value for an event i, APIᵢ, the EEG scaling factor (and in particular, in the above example, the standardised EEG scaling factor API_{EEG,i}) is compared against heart rate change (and in particular, in the above example, the standardised heart rate change API_{HR,i}), with the heart rate change being used to modulate the EEG scaling factor to account for cases of extreme outliers.

One method of achieving the above modulation is a segment modulation approach. To perform this modulation, the plot of API_{EEG} vs API_{HR} is divided into three segments, and each API_{EEG,i} value is modulated based on its positioning in those segments.

Modulation is also dependent on the total number of events where the API is calculated for the specific infant.

The complete segment modulation procedure is defined by the following equation (Note: The general notation I|_{(f*a,b))} below denotes a value of 1 when the operation f(a,b) is True, or 0 otherwise):

$${API}_{i} ( {x}_{i} , {y}_{i} ) = \{ \begin{matrix}{y}_{i} - \frac{{y}_{i} - g ( {x}_{i} )}{{\sum }_{j = 1}^{N} 1 {\vert}_{{y}_{j} > g ( {x}_{j} )}} & if {y}_{i} > g ( {x}_{i} ) \\ {y}_{i} & if h ( {x}_{i} ) \leq {y}_{i} \leq g ( {x}_{i} ) \\ {y}_{i} + \frac{( h ( {x}_{i} ) - {y}_{i} ) ( {\sum }_{j = 1}^{N} 1 {\vert}_{{y}_{j} < h ( {x}_{j} )} )}{N} & if {y}_{i} < h ( {x}_{i} )\end{matrix} \qquad ( 9 )$$

where:
xᵢ=API_{HR,i} (for event i)
yᵢ=API_{EEG,i} (for event i)
g(x)=a first threshold function
h(x)=a second threshold function
N=total number of infant's events

The first and second threshold functions can be template specific. In the specific example described herein, two templates are used, a term template which is derived from data recorded from term infants (aged 37-42 weeks), and a preterm template which is derived from data recorded from preterm infants (aged 29-34 weeks). In that example, g(x)=1.25x+1.5 (for term template) or g(x)=1.25x+5 (for preterm template) and h(x)=0.5x−2 (for term template) or h(x)=0.5x−4 (for preterm template). Other threshold functions may be used, e.g. if other templates are used.

The net effect of the use of threshold functions is that the APIᵢ(xᵢ,yᵢ) score (i.e. the API score for an event i) is set as equal to the API_{EEG,i} score (i.e. yᵢ) if yᵢ is between the two heart rate dependent functions h(x) and g(x). Otherwise, APIᵢ(xᵢ,yᵢ)=yᵢ either increased or decreased by an amount dependent on one of the functions h(x), g(x), depending on whether yᵢ is above g(x) or below h(x).

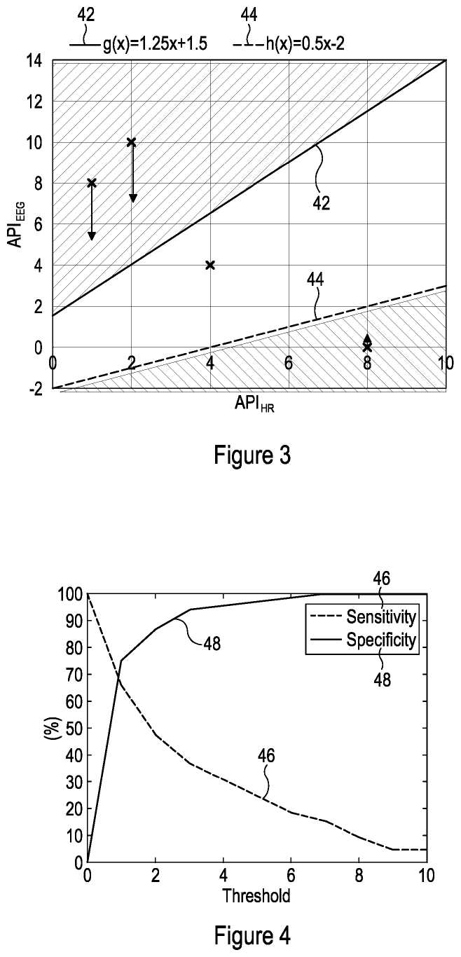

FIG. 3 shows a graphical illustration of the segments and how specific points are modulated according to their positioning for the term template. In this segment modulation, API_{EEG,i} scores are:
a) Modulated down if they are in the top segment, by a proportion equal to the inverse of the number of API_{EEG,i} values for this infant which are in the top segment. In this case, 2 of the API_{EEG,i} values are in the top segment so scores are modulated down by 0.5 the distance to the segment edge, g(x).
b) Modulated up if in the bottom segment, by the proportion of the API_{EEG,i} values for this infant which are in the bottom segment. In this case, 1 API_{EEG,i} value out of 4 is in the bottom segment so modulation is 0.25 the distance to the segment edge, h(x).
c) Unchanged if in the middle segment.

These (undiscretised) values for APIᵢ are then discretised into an API scale (range 0-10) by grouping the data into one of 11 bins based on the following ranges:

| APIᵢ | 0 | 1 | 2 | 3 | 4 | 5 | 6 | 7 | 8 | 9 | 10 |
|---|---|---|---|---|---|---|---|---|---|---|---|
| APIᵢ | <0.5 | 0-0.5 | 0.5-1 | 1-1.5 | 1.5-2 | 2-2.5 | 2.5-3 | 3-3.5 | 3.5-4 | 4-4.5 | >4.5 |
| (undiscretised) | | | | | | | | | | | |

The API thus provides a measure of the strength of a subject's response to an event, such as a tactile or noxious event. A clinician treating the subject may use the API score to determine objectively how strong the subject's response is (e.g. how much pain the subject is experiencing). As an example, using the API scale, pain could be broadly classified by a clinician according to the following categories:
no pain (0),
mild pain (1-3),
moderate pain (4-8)
severe pain (9-10)

The clinician may thus use the API score which is determined for a particular subject as a measurement of the pain felt by the subject in response to the event. This may assist the clinician in making a diagnosis as to what might be the cause of the subject's pain. For example, where the subject has a strong response (e.g. 4 or above) to a purely tactile or mild noxious event that would not be expected to cause pain in a healthy subject, this may indicate to the clinician that the subject has an underlying condition increasing their pain sensitivity.

The threshold between pain and no pain was quantified as the value of the API which provided optimal sensitivity and specificity to discriminate pain from no pain by comparing the API following noxious heel lances and non-noxious control heel lances.

FIG. 4 shows sensitivity 46 and specificity 48 for discriminating ‘pain’ from ‘no pain’ at different thresholds of the API score, calculated from the ability to discriminate a control heel lance from a heel lance response in 65 term infants.

### Template Selection

Brain activity varies dependent on age of a subject. This is particularly the case for infants, as brain development is still ongoing, especially for pre-term infants. Rather than having a single template which is indicative of pain-evoked EEG activity, we have found it beneficial to provide a plurality of templates, which may be age-dependent templates. The EEG template used in the method described above may then be selected from a set of age-dependent templates based on the age of the subject.

In this document we apply two distinct templates for noxious-evoked brain activity, the first derived from infants from 29-34 weeks' postmenstrual age (“preterm template”), and the second derived from infants 37-42 weeks' postmenstrual age (“term template”), allowing a developmentally sensitive age-adjusted API to be created. The derivation of the preterm template is detailed below as an example.

It will be appreciated that more than two templates could be used if required. Indeed any number of templates could be used, each template corresponding to a different age range within the same methodology detailed here.

The template used in the API_{EEG} calculation may be selected strictly based on the age of the subject. That is, an infant having a postmenstrual age of between 29-34 weeks may have an API score calculated using the preterm template, whilst an infant having a postmenstrual age of 34-42 weeks (or older) may have an API score calculated using the term template, which has been validated in infants from 34 weeks.

Alternatively, we have found that a weighted approach for template selection can give better results, in that it is able to account for infants with differing rates of brain maturation.

Two different weighted template selection approaches are described below. The first uses a data-driven approach.

As noted above, we have derived two distinct templates that could be used to calculate the API, depending on the age of the infant. However, using a single age cut-off to choose which template to use is inaccurate as there are intrinsic errors with the estimation of the baby's age. Furthermore, if a baby is deemed ‘dysmature’ (i.e., a delayed brain development for its given age), this would also introduce some discrepancies.

Consequently, we introduce the following ‘fuzzy-data-driven-weighted voting’ approach to ultimately choose which template to use. Note that this voting approach is performed using all events for the specific infant such that the same template will be selected across all events.

If the baby has an age that falls within a first age range, in this case <32 weeks' gestation, the derived preterm template is projected onto the acquired EEG data (using the process described under “template projection” above). Similarly, if the baby has an age that falls within a second age range, in this case >35 weeks' gestation, the corresponding term-age template is projected onto the acquired EEG data. A standardised EEG scaling factor API_{EEG,i} is then calculated as discussed above, and modulated with API_{H}a to produce the final API score.

However, if the baby's age lies in a boundary age range, in this case the range 32-35 weeks' gestation, we apply the template projection twice, once for each template, resulting in two values: API_{EEG,i}^{l} and API_{EEG,i}^{h} where l and h denote the low-age (preterm) and high-age (term) templates, respectively.

To select which set of template-derived metrics to then use (for babies whose age lies in the range 32-35 weeks' gestation), we apply a data-driven-weighted approach, multiplying API_{EEG,i}^{l} and API_{EEG,i}^{h} by a weighting determined from data-driven scaling functions across age (as shown in FIG. 6). This weighting provides a measure of the prior uncertainty on the appropriateness of that template for the specific age of the infant. Denoting the infant's age as a and the data-driven scaling functions as Sₗ(a) and Sₕ(a), respectively, the selected API_{EEG,i} is chosen as follows:

$${API}_{EEG , i} = \{ \begin{matrix}{API}_{EEG , i}^{i} & if {Λ}_{\hat{a}} < 0 \\ {API}_{EEG , i}^{h} & if {Λ}_{\hat{a}} > 0\end{matrix} \qquad ( 10 )$$

where:
λ_{â}=Σⱼ₌₁^{N} (API_{EEG,j}^{h} Sₕ(â)−API_{EEG,j}^{l}Sₗ(â))
N=total number of infant's events
â=Age of infant

The voting function and age boundaries of 32 and 35 weeks' gestation were determined in a training set of 13 infants and tested in an independent sample of 17 infants. The data-driven scaling functions were determined using data from 96 infants aged 28-42 weeks' gestation with recordings in response to a clinically-required heel lance. A machine learning approach was used, fitting Gaussian Processes (GPs) to model the development of the preterm and term template responses across age. We utilized a GP as it is a non-parametric approach and so we needed to make no prior assumptions on the form that the scaling functions should take.

The fits from the GP were then scaled according to the maximum and minimum values across the age range to identify the weighting for each template according to postmenstrual age, and to identify the transition between the two templates.

The selected API_{EEG,i} is then carried forward to the rest of the API calculation.

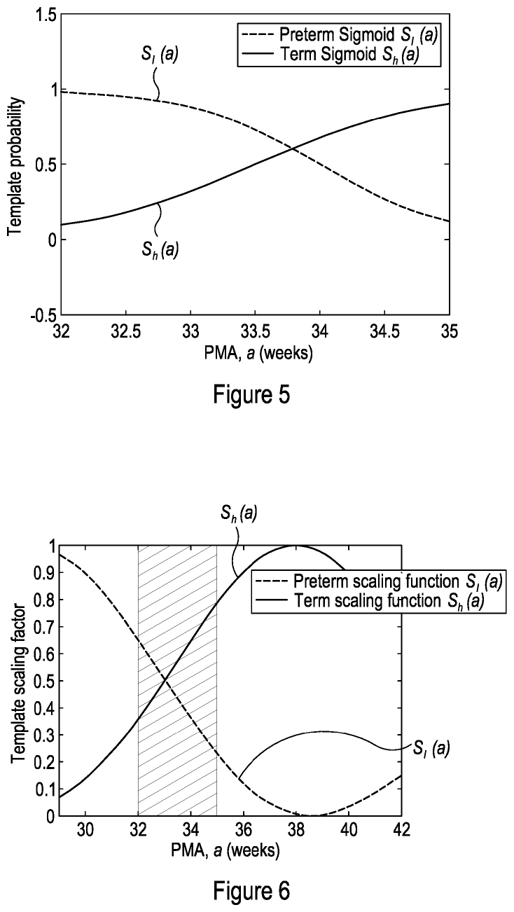

As an alternative to the data-driven weighting approach used above, Sigmoid functions may be used instead of the data-driven scaling functions as Si(a) and Sₕ(a) discussed above. Illustrative Sigmoid weighting functions are shown in FIG. 5.

It will be appreciated that any given template may be derived for a specific age group, and thus the age ranges described above are template dependent. The first and second age ranges, as well as the boundary age range, may therefore change dependent on the templates that are used. Further, if more than two templates are used there may be more than two age ranges, and more than one boundary age range. Thus a plurality of voting functions may be used in template selection.

### API Summary

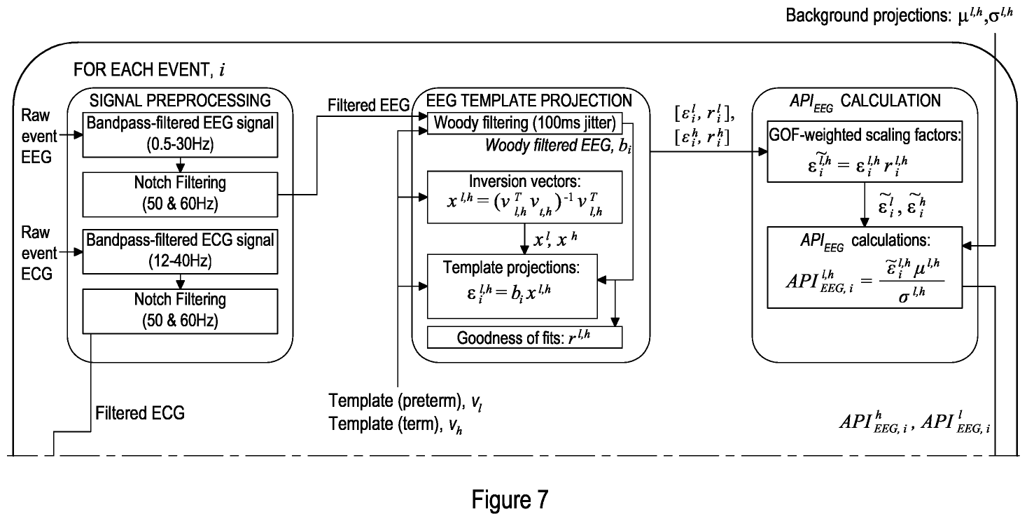

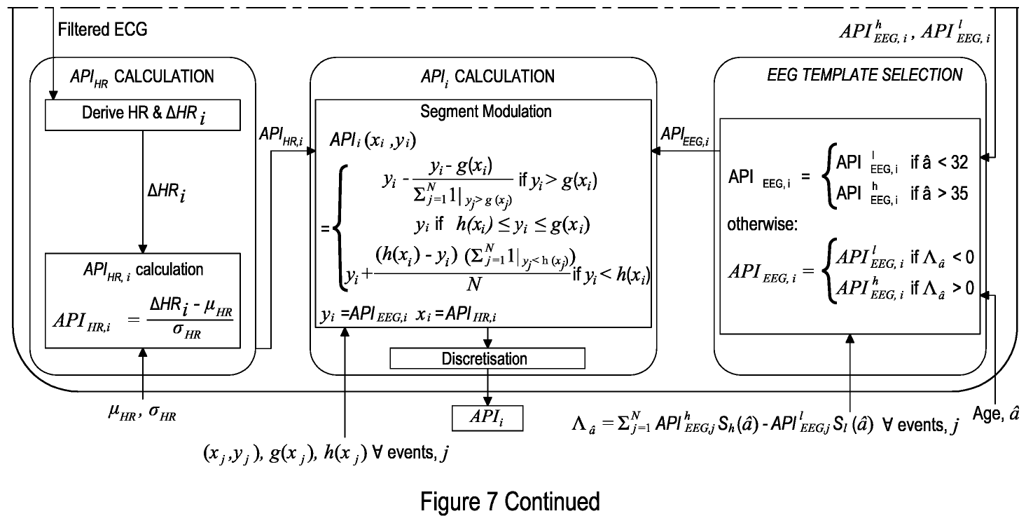

A full summary of the API calculation algorithm is shown in FIG. 7. All formulae are presented, accounting for the use of both templates (prior to template selection), where appropriate.

The output of the algorithm is a score, API, which provides an invariant measure of the pain felt by an infant in response to an event. Such a score allows a clinician to assess whether a subject is feeling pain, how much pain the subject is feeling, and can allow meaningful comparisons to be drawn between individuals. The API score thus constitutes a useful tool for clinicians wanting to determine what could be the underlying cause of an infant's pain.

### Deriving a Template of Noxious-Evoked Brain Activity for 29-34 Weeks Postmenstrual Age (PMA)

EEG activity in response to a noxious stimulus (clinically-required heel lance), non-noxious stimulus (control heel lance) and in background EEG were compared in infants aged 29-34 weeks' PMA at the time of study. Infants were split into a data set used to derive the template (13 infants) and a test set (17 infants) used for validation (see below).

### 1. Deriving the Preterm Template Using PCA

To characterise the noxious-evoked template at the Cz electrode, data was first filtered from 0.5-8 Hz, with a notch filter at 50 Hz, extracted from the recordings in 2.5 second epochs, with 1 second before the stimulus, and baseline corrected to the pre-stimulus mean. The data was then Woody filtered with a maximum jitter of 50 ms in the time window 300-650 ms following the stimulus/background annotation (time window chosen from visual inspection of the data) to achieve maximum correlation between the individual traces and the data average. Principal Component Analysis (PCA) was then conducted on the responses to the noxious stimulus, non-noxious stimulus and background activity in the time window 300-650 ms following the stimulus/background annotation. The first two PCs accounted for 89% of the variance within the data and were the only ones considered. The weights of the PCs were compared across stimuli using a repeated measures ANOVA (Analysis of Variance). Post hoc comparisons of pairs were corrected for multiple comparisons using Holm's method. The component with significantly higher weights in response to the noxious stimulus compared with the non-noxious stimulus and in background data was selected as the template of noxious-evoked brain activity.

### 2 Validating the Preterm Template

To validate the new template, we aimed to determine whether the brain activity characterised by the template was specific to noxious stimuli in independent infants. In 17 infants aged from 29-34 weeks' gestation who received a clinically required heel lance and control heel lance, the magnitude of the template response was calculated at the Cz electrode by projecting the template onto the data in the time window 300-650 ms after the stimulus. Data was first Woody filtered with a maximum jitter of 100 ms to achieve maximum correlation between the individual traces and the template. The magnitudes were compared using a paired t-test.

A subset of 12 infants also received visual (a flash of light), tactile (a tendon hammer applied to the heel) and auditory (a tone) stimuli. The data from these trials was similarly Woody filtered with a maximum jitter of 100 ms and the template projected onto the data in the time window 300-650 ms after the stimulus at the Cz electrode. The magnitudes were compared using a linear mixed effects model, with stimulus type taken as a fixed effect and subject index as a random effect.

FIG. 8 illustrates development and testing of the preterm template. In FIG. 8A principal component analysis was conducted at the Cz electrode 300-650 ms after stimulus (grey boxes) to derive the template, by comparing background brain activity with activity evoked by noxious and non-noxious stimulation. The Woody-filtered EEG 50 is shown overlaid with the template 52 (in black). The magnitude of template activity was significantly higher after noxious compared with non-noxious stimulation and background activity and was therefore considered to be evoked by the noxious stimulation. In FIG. 8B average and Woody-filtered EEG shown in background EEG and in response to auditory, visual, tactile, non-noxious control and noxious (heel lance) stimulation in an independent test set. Again, the Woody-filtered EEG 50 is shown overlaid with the template 52 (in black). The magnitude of template activity was significantly higher after noxious stimulation compared with the other stimuli (**p<0.01; error bars indicate means f SEM).

### Results: Evidence of the Validity and Potential Clinical Applications for the API

[Note: In the results below, we have replaced the use of the methods notation APIᵢ with simply API for clarity and the distinction between events is now implied]

1. Noxious-Evoked Brain Activity was Characterised in Infants from 29-34 Weeks' Gestation.

A novel template of noxious-evoked brain activity in infants from 29-34 weeks' gestation was identified by contrasting the patterns of brain activity evoked by noxious and non-noxious stimuli in 13 infants. The weights of the first principal component (which accounted for 55% of the variance within the data) were significantly higher in response to the noxious stimulus compared with the non-noxious stimulus and in background brain activity (p<0.005, FIG. 8A) and this component was defined as the template of noxious-evoked brain activity for this age range in subsequent analysis. There was no significant difference between the weights of the second principal component, which accounted for 34% of the variance, across modalities (p=0.46).

The validity of this template was next tested in an independent sample of 17 infants. Projecting the template onto activity evoked by the noxious stimulus showed a significantly higher response than that observed following the non-noxious control heel lance (p=0.00015, FIG. 8B) and in the background activity (p=0.0004, FIG. 8B). A subset of 12 infants also received visual, auditory and tactile stimuli. These stimuli did not evoke activity, characterised by the template, that was significantly higher than background activity (p>0.05, FIG. 8B).

Having validated the new template of noxious-evoked brain activity for use in preterm infants from 29-34 weeks' gestation we then confirmed that the inclusion of the new template in the full API method (projection of the template, scaling by goodness-of-fit, normalisation by background activity, modulation by API_{HR}) was valid at this age range. In the full sample of 30 infants, the median API following the heel lance was 6 (interquartile range=7). Following the control heel lance the median API was 0 (2), indicating no pain.

### Performance of the API

The API score discussed above has increased performance compared against use of an EEG template scaling factor that is not modulated by heart rate change and/or standardised using background data and/or weighted using goodness-of-fit.

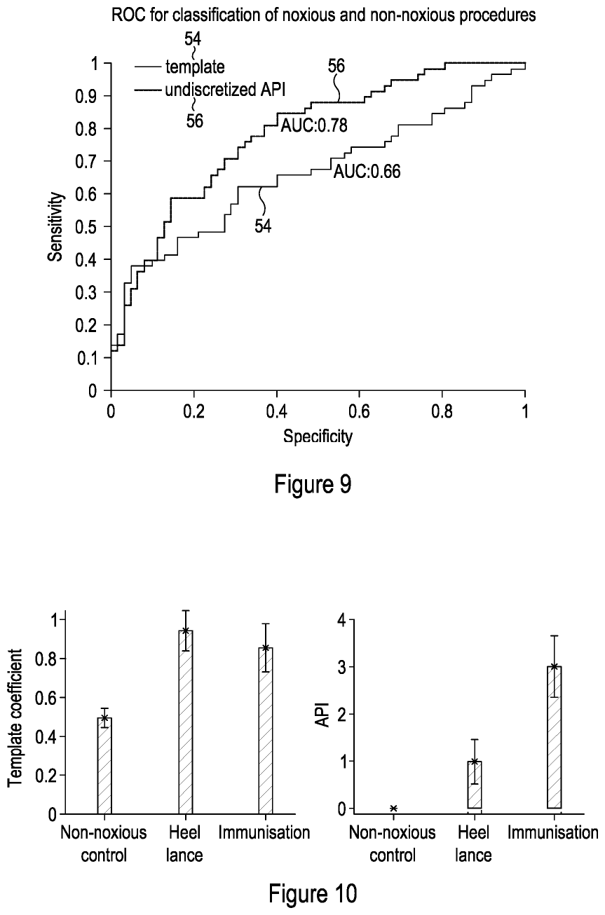

Sensitivity and specificity of the API (undiscretized) scores were calculated by comparing the responses between a noxious procedure (heel lance) and a non-noxious control in a sample of term neonates. Receiver operating characteristic (ROC) curves were produced using a classification by logistic regression method and the area under the curve (AUC) calculated as a measure of discrimination performance between a painful and non-painful stimulus. This is shown in FIG. 9, which illustrates ROC curves for the discrimination between noxious and non-noxious stimuli. ROC was performed for the EEG template scaling factor alone 54 and for the undiscretised API 56 (n=120, 58 participants receiving a clinically required heel lance and 62 participants receiving a non-noxious control. Area under the curve (AUC) for the undiscretised API is 0.78 and for the template scaling factor it is 0.66). The AUC of 0.78 represents a 18% increase compared with the EEG template approach alone.

### The API Characterises Different Types of Clinical Procedures.

Heel lances and injections are amongst the most common acute painful procedures that neonates are exposed to during hospitalisation.

FIG. 10 shows average EEG template scaling factors (left) and median API scores (right) in response to common acute clinical procedures and a non-noxious control (non-noxious control n=62, heel lance n=58, immunisations n=25). The API scores reflect pain intensity caused by different procedures. Error bars indicate means f SEM (left) and medians f standard error of the median (right). It can be seen that the median API scores calculated following these procedures is consistent with the intensity of each procedure (e.g. immunisation median API=3, heel lance median API=1) and allows a better discrimination between acute clinical procedures compared with the template EEG coefficients alone.

Evidence that the API is Reduced by Analgesic Administration

### 1. Topical Local Anaesthetic Application Reduces the API

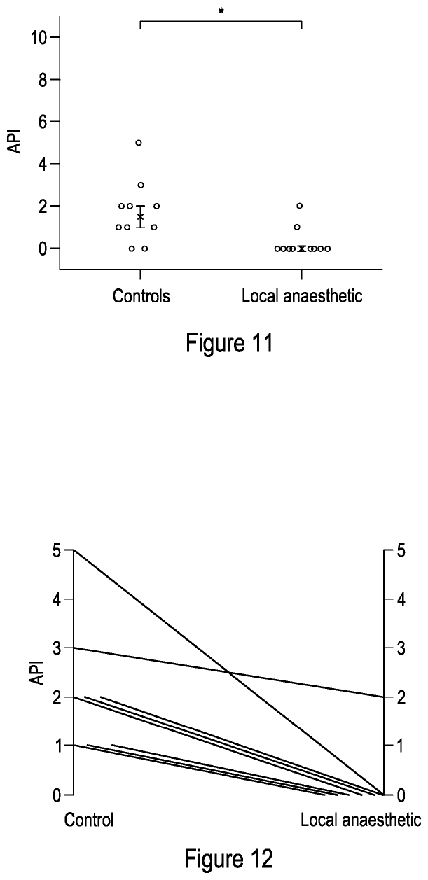

FIG. 11 shows API scores following experimental noxious stimulation applied to a group of neonates (n=10 participants) without anaesthetic (Control) and with local anaesthetic applied prior to the stimulation (Local anaesthetic). API scores are significantly lower in the local anaesthetic group (*p=0.023, Wilcoxon signed rank test) Error bars indicate median±standard error of the median. Thus, as demonstrated in FIG. 11, application of topical local anaesthetic (prior to cannulation) significantly reduces the API in response to the experimental noxious stimulation with and without treatment (without local anaesthetic median API=1.5 (IQR:2-1); with local anaesthetic median API=0 (IQR:0-0), Wilcoxon signed rank test p=0.023).

2. API is Reduced in Individual Infants after Topical Local Anaesthetic Application

FIG. 12 shows individual neonates' API scores in response to experimental noxious stimulation applied before and after the application of local anaesthetic (n=8 participants). Neonates at rest with API≥1 are included in this figure. FIG. 12 clearly demonstrates that individual infants at rest who had an API≥1 (indicative of mild, moderate or severe pain) had a reduced API after topical local anaesthetic was applied to the surface of the skin.

### 3. Paracetamol Reduces the API Evoked by Immunisation

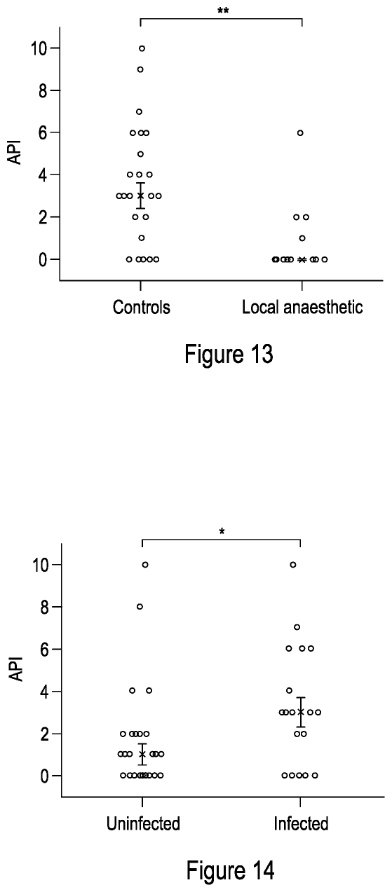

FIG. 13 shows that API scores following immunisations are significantly lower in a group of neonates who received paracetamol compared with a group of infants who did not receive paracetamol prior the immunisations. Control Group n=15 participants, Intervention Group n=14 participants, error bars indicate median±standard error of the median (mixed effects model for ordinal data with subject set as random effect **p=0.004).

A cohort of 22 ex-premature neonates was studied during the administration of routine immunisations in the neonatal unit. The Control Group includes 12 neonates who received routine immunisations with no prior analgesic medication. During the study the local guidelines were updated to administer paracetamol (15 mg/kg) 1-hour prior to the MenB vaccine. Ten neonates studied following the guideline change constitute the Intervention Group. EEG and heart rate were acquired, and the API calculated following the methods described above. As shown in FIG. 13, administration of paracetamol 1 hour prior to immunisation significantly reduces the API evoked by the immunisation compared with infants who did not receive paracetamol (Control Group median API=3 (IQR:5.75-1.25); Intervention Group median API=0 (IQR:0-0); p=0.004, mixed effects model for ordinal data with subject set as random effect).

### The API in a Neonate at Rest Relates to Subsequent API Responses to Painful Events and can Drive Treatment Options

1. A High API Score at Rest Suggests that Clinical Procedures Will Evoke a High API.

API was measured in a sample of 15 term neonates at rest by applying mild experimental noxious stimulation to the foot before a clinically required heel lance. The API scores in response to the clinical procedure are significantly correlated with the API score at rest (p=0.018, R²=0.36, Spearman's linear correlation), suggesting that API at baseline can be used to infer individual neonates' responses to acutely painful procedures.

### The API is Sensitive to Infant Wellbeing

### 1. Infection Data

FIG. 14 shows that neonates with infection in the first days of life (infected group, n=19 participants) present significantly higher API scores compared with healthy neonates (uninfected group, n=28 participants), (Wilcoxon rank sum test *p=0.017). Error bars indicate median±standard error of the median.

A sample of 47 term neonates presenting risk factors and clinical signs of early onset neonatal sepsis were screened for suspected infection and studied during a clinically required heel lance to assess infection markers. As shown in FIG. 14, neonates who have an infection (identified as those infants with a C-reactive protein (CRP) level>=10 and receiving antibiotic treatment for suspected sepsis) have a greater API (median API=3, IQR:5.5-0.5) than neonates with a CRP level<=10 (median API=1, IQR:2-0) where antibiotic treatment is discontinued (Wilcoxon rank sum test p=0.017). This demonstrates the application of the API as a measure of pain sensitivity during common conditions like infection and can be used to assess pain during other conditions like post-surgery.

### Multi-Site Reproducibility

This work has been reproduced by other research groups at the Royal Devon University Healthcare NHS Foundation Trust.

We have described herein a brain-derived clinical neonatal pain assessment tool (the Acute Pain Index, API) that can be used to infer pain intensity in individual neonates and infants from 29 weeks' gestation to 6 months of age. The API can be calculated during a baseline rest period to infer underlying pain sensitivity and in response to acutely painful procedures.

It will be appreciated that the approach discussed above can be applied to subjects in other age ranges, such as older infants, children and adults.

The API as discussed above is calculated using a machine-learning approach to analyse EEG and ECG signals that are evoked in response to a sharp tactile (mild noxious) stimulus applied to the surface of the skin while the infant is at rest, or by an acute painful clinically-required procedure. The API is calculated by first identifying an age-dependent pattern of noxious-evoked EEG activity recorded in response to stimulation in the form of a template of EEG activity in response to such a stimulus that is derived by analysis of data from a plurality of subjects. That template is then used to characterise an individual's response to a similar stimulus by scaling the template to fit EEG data recorded from that individual. Normalising this output using background EEG activity permits the measure to be standardised across individuals. Further, modulating the normalised EEG output using heart rate data can improve the reliability of the measure.

It will be appreciated that the same template-based approach could be taken to quantifying the response of a subject to other events than noxious and/or tactile stimuli, such as visual or auditory stimuli.

1. A method comprising:
acquiring, using an EEG monitoring system, EEG data from a subject, said data being recorded over a first time period, said first time period including an event;
acquiring, using a heart rate monitoring system, heart rate data from the subject, said heart rate data being recorded over a second time period, said second time period also including the event;
scaling an EEG template to fit event EEG data at a specified latency following the event to derive an EEG scaling factor;
determining a heart rate change due to the event using the heart rate data; and
combining the EEG scaling factor and the heart rate change to generate a score indicative of a response of the subject to the event.

2. The method of claim 1, the method further including:
selecting the EEG template from a set of age-dependent templates to obtain an age-appropriate EEG template, said selection being based on an age of the subject.

3. The method of claim 2, wherein the selection is made using a weighted probability function.

4. The method of claim 1 or claim 2 or claim 3, the method further comprising:
deriving a goodness-of-fit between the EEG template and the event EEG data; and
weighting the EEG scaling factor using the goodness-of-fit to produce a weighted EEG scaling factor.

5. The method of any preceding claim, the method further comprising:
acquiring, using the EEG monitoring system, baseline EEG data from the subject, said baseline EEG data being recorded over a plurality of baseline EEG time periods prior to the event; and
modulating the EEG scaling factor using the baseline EEG data to obtain a modulated EEG scaling factor.

6. The method of claim 5, wherein modulating the EEG scaling factor comprises:
scaling the EEG template to fit the baseline EEG data to produce scaled baseline EEG data for each of said baseline EEG time periods; and
modulating the EEG scaling factor using the scaled baseline EEG data to obtain the modulated EEG scaling factor.

7. The method of claim 5 or claim 6, wherein the method further comprising:
deriving, for each of the plurality of baseline EEG time periods, a goodness-of-fit between the EEG template and the scaled baseline EEG data;
weighting, for each of the plurality of baseline EEG time periods, the scaled baseline EEG data using the derived goodness-of-fit to produce weighted scaled baseline EEG data;
calculating the mean and the standard deviation of the weighted scaled baseline EEG data; and
standardising the EEG scaling factor using:
$${API}_{EEG , i} = \frac{{ϵ}_{i} {r}_{i} - μ}{σ}$$
where ϵᵢ is the EEG scaling factor for the event, rᵢ is the goodness-of-fit between the EEG template and the event EEG data, μ is the mean of the weighted scaled baseline EEG data, and σ is the standard deviation of the weighted scaled baseline EEG data.

8. The method of any preceding claim, further comprising:
acquiring, using the heart rate monitoring system, baseline heart rate data from the subject, said baseline heart rate data being recorded over a plurality of baseline heart rate time periods prior to the event; and
modulating the heart rate change due to the event using the baseline heart rate data.

9. The method of claim 8, wherein the method further comprises:
determining a pre-event heart rate change for each of the plurality of baseline heart rate time periods; and
standardising the heart rate change due to the event by subtracting the mean of the pre-event heart rate changes from the heart rate change due to the event and dividing the result by the standard deviation of the pre-event heart rate changes.

10. The method of any preceding claim, wherein the step of combining the standardised EEG scaling factor and the standardised heart rate change to generate a score comprises:
defining a first threshold function based on the standardised heart rate change and a second threshold function based on the standardised heart rate change,
leaving the score unchanged if the standardised EEG scaling factor falls between the first and second threshold functions,
decreasing the score if the standardised EEG scaling factor is greater than the first threshold function, and
increasing the score if the standardised EEG scaling factor is less than the second threshold function.

11. The method of any preceding claim, further comprising discretising the score.

12. The method of any preceding claim, wherein the event is a tactile stimulus and/or a noxious stimulus.

13. A system for quantifying pain experienced by a subject in response to an event, the system comprising:
a processor operable to carry out the method of any one of claims 1 to 12 using data acquired by an EEG monitoring system and a heart rate monitoring system.

14. The system of claim 13, further comprising:
an EEG monitoring system operable to acquire EEG data from the subject; and
a heart rate monitoring system operable to acquire heart rate data from the subject.

15. A computer programme product operable, when run on a processor of a system according to claim 13 or claim 14, to cause the processor to carry out the method of any one of claims 1 to 12.
